(19) United States
(12) Patent Application Publication
(10) Pub. No.: US 2008/0166265 A1
(43) Pub. Date: Jul. 10, 2008

(54) Method and system for the transformation of molecules, this process being used to transform waste into useful substances and energy

(76) Inventor: Andrew Eric Day, Longmeadow, MA (US)

Correspondence Address: Andrew Eric Day;Eric Day, 325 Williams St., Longmeadow, MA, 01106, US

(21) Appl. No.: 11/621,801

(22) Filed: Jan. 10, 2007

(51) Int. Cl. B09B 3/00; A61L 9/00

(52) U.S. Cl. 422/30; 422/184.1; 423/645

(57) ABSTRACT

The system is based on a recirculating Carbon Flow Loop, within which toxins in municipal waste or other feedstock are neutralized in a plasma reactor, by using an electric arc in ionized gas to generate ultra high temperatures. This breaks down substances into their basic molecules, and transforms the feedstock into syngas (which is comprised predominantly of hydrogen and carbon monoxide). This can be processed by a water shift reactor, an engine driven electric generator or another exothermic device where carbon monoxide is transformed into carbon dioxide. This continues flowing in the carbon loop to an Algae Bioreactor. Here photosynthesis of the algae transforms the carbon dioxide to become part of an oil rich carbohydrate. This can either continue to the next stage as feedstock and recirculate again around the Carbon Loop and/or exit, and be used to manufacture biofuel or other substances.

### FIELD OF INVENTION

The planet is being poisoned by toxic waste, while waste is not being put to useful work:

1. Carbon Dioxide emissions from combustion engines, (used in Power Stations etc.) and rotting waste are creating global warming gasses. This could contribute to destroying the planet, as we know it. The process may soon be irreversible.

2. Toxic waste from industrial factories and landfills is finding its way into our ground water supply.

3. Medical waste and dangerous bacteria need to be completely destroyed.

4. Landfills release methane into the atmosphere. Methane is 23 times more effective over a 100 year period at trapping heat as Carbon Dioxide.

5. Landfills and other waste streams are not being utilized as a resource.

The need to address these problems is urgent and compelling.

It is known that Photosynthesis of Algae creates Carbohydrates by combining Carbon Dioxide with Hydrogen. Plasma converters break down substances to their basic molecules by exposing them to the very high temperatures of an electric arc in ionized gas. Hydrogen engines release energy for useful work and steam as the exhaust gas

This invention is a system, which uses these processes and heat recovery techniques to form an efficient and practical way of cleaning up toxic waste and other refuse. By using landfills and other waste streams as a recoverable energy source we reduce our dependency on petroleum oil.

### BACKGROUND OF INVENTION

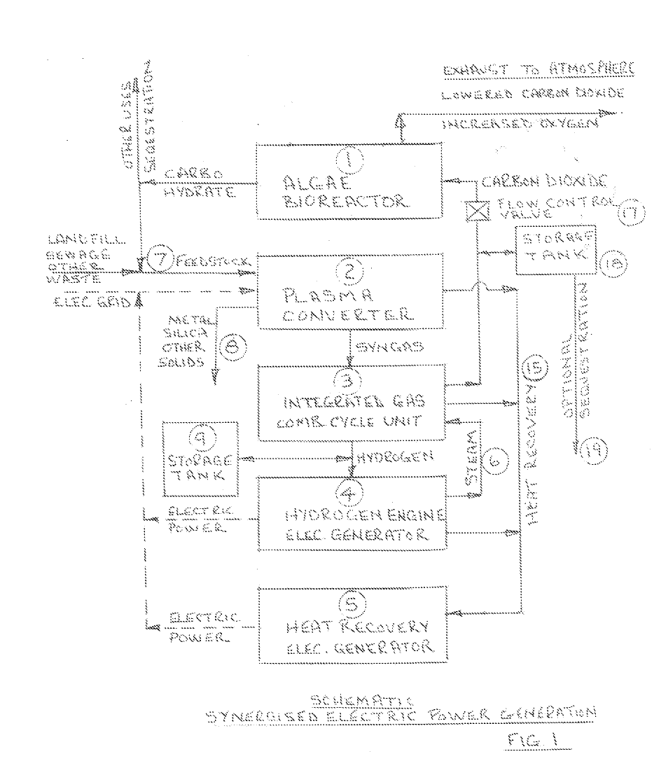

Building blocks for this system as shown in FIG. 1 are known:

1. Algae Bioreactors use fast growing Algae, that in the presence of sunlight feed on Carbon Dioxide CO₂, to become a valuable source of Carbohydrate. Carbon Dioxide is thus converted from a global warming pollutant into useful fuel feedstock rich in Hydrogen. Where up to 80% absorption is targeted i.e.

Carbon Dioxide+Water+Plus sunlight=Glucose+Water+Oxygen 6 CO₂+12 H₂O+Plus sunlight C₆ H₁₂ O₆+6 H₂O+6O₂

In general terms this is as follows:

Carbohydrate+Water+Oxygen n CO₂+2nH₂+ATP+NADPH−(C H₂O)n+n H₂O+nO₂

Where n is defined according to the structure of the resulting carbohydrate,

ATP is adenosine triphosphate,

NADPH is nicotinamide adenosine dinucleotide phosphate.

Hydrocarbons

Hydrocarbons which typically are defined as CnH₂n+₂ lack Oxygen.

2. Plasma Converters achieve temperatures hotter than the sun's surface, by striking an electric arc though ionized gas, much in the same way as a lightning bolt. At these elevated temperatures, molecules within compounds are converted into basic substances. Hydro Carbons and Carbohydrates split into Carbon Monoxide and Hydrogen. Base metals, some solid Carbon and silica form part of a molten discharge. This can be drained off to solidify on cooling to become a source for precious metal and silica. The non-precious slag can be used as a building material for industrial products.

The Plasma Converter output is Syngas.

The active gasses are mainly Carbon Monoxide CO and Hydrogen H₂

3. Integrated Gasification Combined Cycle units are used to combine high temperature steam with the Syngas. This combines Oxygen with Carbon Monoxide to become Carbon Dioxide, and bleeds off the remaining Hydrogen gas, before feeding the Carbon Dioxide gas back to the Algae Bioreactor.

i.e.: Syngas+Steam=CarbonDioxide+Hydrogen (CO+H₂)+H₂O═CO₂+2H₂

4. Hydrogen Engines ignite the Hydrogen in the engine combustion chamber and can be used to drive an electric generator or other devices. The exhaust “gas” from this process is a ready source of steam, which can be fed directly to the Integrated Gasification Combined Cycle unit, or after recovering the heat energy, stored as water.

5. Heat Recovery from the Plasma Converter, the Converter molten discharge, the Integrated Gasification Combined Cycle unit, and the Hydrogen Engine can be used for many industrial processes, including a refrigerant turbine to power an electric generator. This unit uses the waste heat to evaporate refrigerant gas. This is used to power a low temperature gas turbine engine, which drives a generator, This is used to supplement the electric power provided by the Hydrogen Engine.

### OBJECT OF INVENTION

1. It is the objective of the present invention to provide a method and system to generate electricity and/or produce hydrogen gas, using landfill, sewage or other waste streams, while neutralizing toxins in the feedstock, by breaking them down to their base molecules.

2. It is the objective of the present invention to provide a method and system to generate electricity and/or produce hydrogen gas, while limiting Carbon Dioxide greenhouse gas emissions.

3. It is the objective of the present invention to provide a method and system to generate electricity from day to day without interruption.

4. It is the objective of the present invention to provide a method and system to provide a recirculating source of steam for molecular transformation.

5. It is the objective of the present invention to provide a method and system to provide a means of gathering, transporting and harvesting Hydrogen.

### SUMMARY OF INVENTION

It can be seen in FIG. 1, that we have two flow loops, one Carbon and one Hydrogen. The first is a Carbon Loop, by which the Algae Bioreactor (Item 1) gathers and supplies Carbohydrates via the Feedstock Input (Item 7) to the Plasma Converter (Item 2), which supplies Syngas to the Integrated Gasification Combined Cycle Unit (Item 3), which supplies Carbon Dioxide to the Algae Bioreactor (Item 1). This provides an overall means of gathering, transporting and harvesting Hydrogen from the Algae Bioreactor to the Hydrogen Storage tank (Item 9). To Algae Bioreactor Carbon flow as follows:

Carbon to Atmosphere=Carbon to Algae Bioreactor−Carbon flow from Bioreactor

The Carbon Dioxide greenhouse gas emission flowing to atmosphere, can be controlled by measuring the them and adjusting the Carbon Dioxide Flow Limiting Valve (Item 17), as shown in FIGS. 1 through 6. To avoid a build up of Carbon Dioxide in Storage Tank (Item 18) Feedstock Flow to the Plasma Reactor needs also to be adjusted. It can be seen that if Carbon could be removed with other molten solids at the Plasma Converter Discharge Port (Item 8) or by other means, the Feedstock flow rate could be increased and more Hydrogen transferred. Alternatively increased flow rates could be achieved by sequestration of Carbon Dioxide at the Storage Tank reference (Item18) and (Item 19).

As an alternative operation, all or some of the Carbohydrate output from the Algae Bioreactor can be put to other uses, or saved by sequestration storage. This being replaced by another feedstock, from landfill, sewage or other waste, as long as the Algae Bioreactor Carbon balance as discussed above is maintained.

In the steam loop, Hydrogen transfers from the Integrated Gasification Combined Cycle Unit to the Hydrogen Engine, where during combustion and heat release the Hydrogen combines with Oxygen to form steam. The steam is then fed to the Integrated Gasification Combined Cycle Unit, where during heat absorption the steam is converted back to Hydrogen again.

The applicants have formulated an innovative and economical method of converting landfill waste, sewage, and other feedstock waste to provide Hydrogen gas. A Hydrogen, and a heat recovery engine are then used to drive generators to provide electric power. Fuel cells could also be used. By storing some of the Hydrogen, a reserve fuel supply is maintained. The Photosynthesis can only occur during sunlight hours. When Carbon absorption in the Algae Bioreactor is shut down due to lack of sunlight, the Hydrogen engine is operated from the reserve Hydrogen fuel supply. As a backup to this, other energy storage devices could be used. Battery storage, or other potential and kinetic devices are available.

The Algae Bioreactor consumes Carbon Dioxide emissions. In this way Carbon Dioxide (CO₂) greenhouse gasses (GHG) are minimized.

Variations on this proposal can be made to suit specific application. These are shown on FIGS. 1 through 6.

FIG. 1. the features of other optional configurations are listed below:

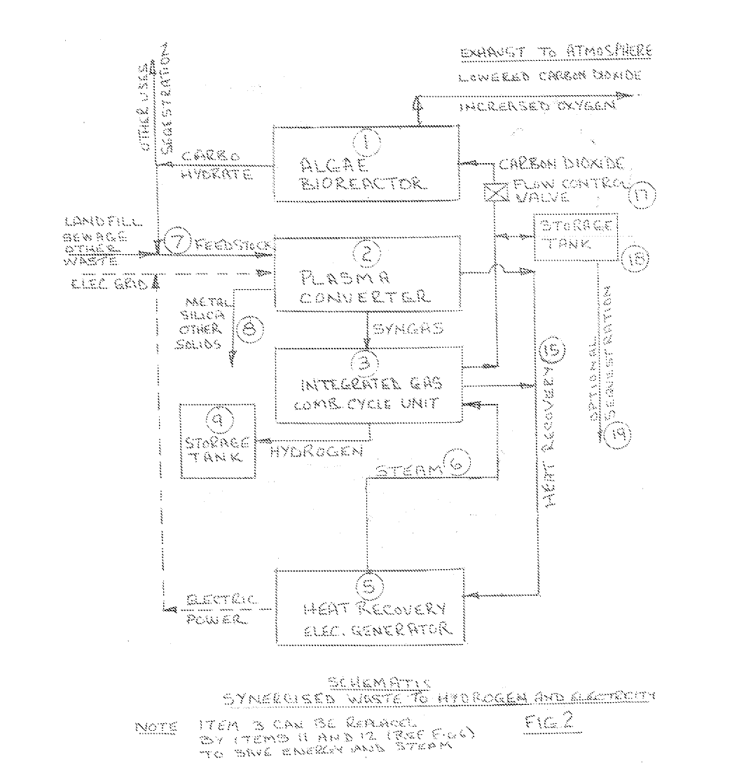

FIG. 2. Less electricity, more Hydrogen, lower cost

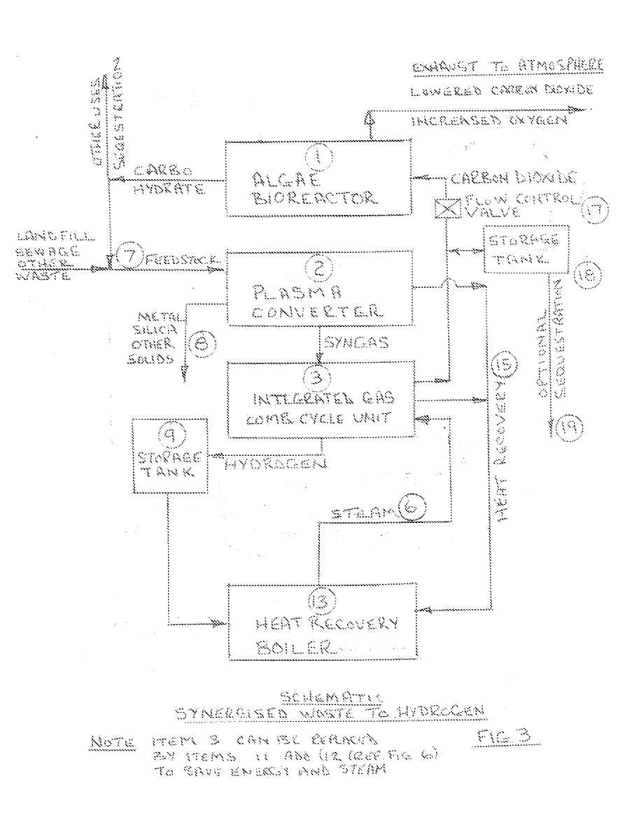

FIG. 3. No electricity, even more Hydrogen, even lower cost

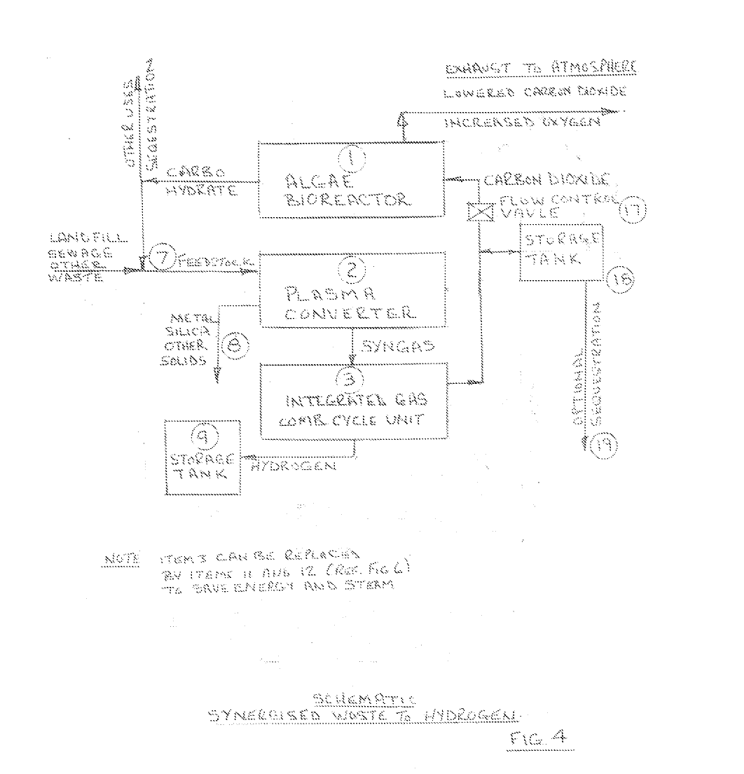

FIG. 4. No electricity, similar Hydrogen, no heat recovery, no steam supply for Integrated Gasification Combined Cycle unit

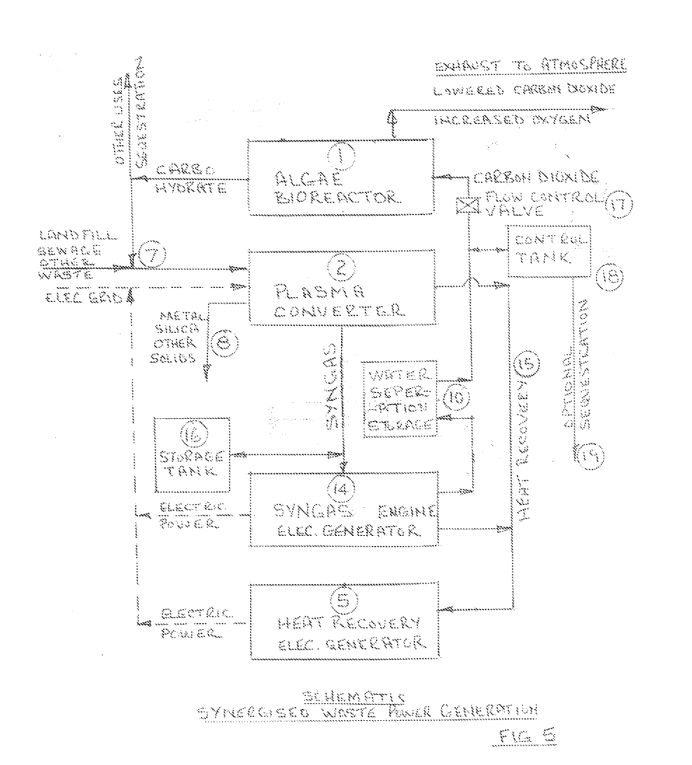

FIG. 5. No Hydrogen production, more electricity

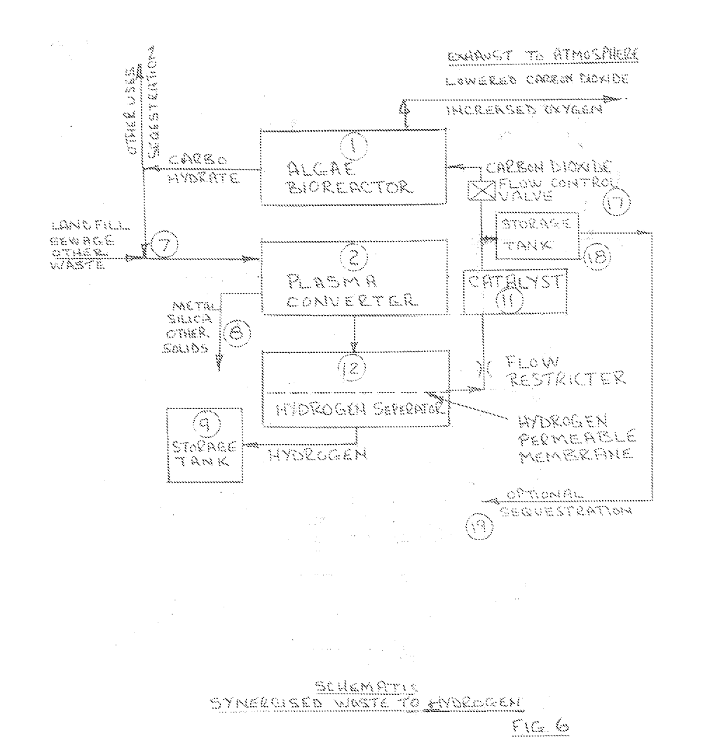

FIG. 6. No electricity, no heat recovery, even lower cost

### DESCRIPTION OF PREFERRED EMBODIMENT

As shown on FIG. 1, Carbohydrate/HydroCarbon or other feedstock (Item 7), plus Carbohydrate from the Algae Bioreactor (Item 1), is fed to the Plasma Converter (Item 2) to produce Syngas. This is then fed to the Integrated Gasification Combined Cycle Unit (Item 3), where with steam input (Item 6) the Carbon Monoxide is converted into Carbon Dioxide and fed back to the Algae Bioreactor (Item 1). Hydrogen is also filtered out and fed to the Hydrogen Engine Electric Generator (Item 4) and Hydrogen Storage Tank (Item 9). With adequate Hydrogen storage the Hydrogen Engine Electric Generator (Item 4) becomes an uninterrupted source of electric power. It is also used to provide hot engine water to the Energy Recovery System (Item 15). The exhaust “Gas” is steam and its used directly by the Integrated Gasification Combined Cycle Unit for molecule processing. Heat can also recovered from the Plasma Converter Molten Discharge (Item 8), and the Plasma Converter and Integrated Gasification Combined Cycle Unit cooling jackets. To improve overall operating efficiency, recovered heat can be used to evaporate refrigerant gas, which powers a low temperature gas turbine engine (Item 5) This drives a generator, which supplements the electric power provided by the Hydrogen Engine Electric Generator. A byproduct of the Plasma Converter (Item 2) operation is the base metals, silica, Carbon, and other solids, which melt and form part of a molten discharge (Item 8). This can be drained off to solidify on cooling and become a source for precious metal recovery. The silica and other products can be recovered as a building material for many industrial products and uses.

As shown on the embodiment in FIG. 2, the FIG. 1 system is modified to omit item 4, the Hydrogen Engine Electric Generator. This embodiment is better suited for applications where more Hydrogen is required (to be stored in item 9) as the final product. Supplemental heat may be required to boil the heat recovery water into steam (Item 6). This embodiment reduces the electric power, which can be supplied to the electric grid, but also reduces the initial capital cost of the system

As shown on the embodiment in FIG. 3, the FIG. 1 system is modified to omit item 4, the Hydrogen Engine Electric Generator and item 5, the Heat recovery Electric Generator.

This is replaced by item13, a heat recovery boiler. This embodiment is suited for applications where only Hydrogen is required (to be stored in item 9) as the final product. This embodiment does not provide any electric power to the electric grid but reduces the initial capital cost of the system.

As shown on the embodiment in FIG. 4, the FIG. 1 system is modified to omit item 4, the Hydrogen Engine Electric Generator, item 5, the Heat recovery Electric Generator, and the Heat recovery System, item 15. It omits steam injection into the Integrated Gasification Combined Cycle Unit. This needs to be replaced by another clean water source. This further reduces the initial capital cost of the system. This embodiment is suited for applications where only Hydrogen is required (to be stored in item 9) as the final product. This embodiment does not provide any electric power to the electric grid but reduces the initial capital cost of the system.

As shown on the embodiment in FIG. 5, the FIG. 1 system is modified to omit item 3, the Integrated Gasification Combined Cycle unit, and item 4, the Hydrogen Engine Electric Generator. These are replaced by item 14, the Syngas Engine Electric Generator, and item10, the engine exhaust gas Water Separator And Storage unit. This embodiment generates electricity but does not provide any Hydrogen gas. It reduces the initial capital cost of the system.

As shown on the embodiment in FIG. 6, the FIG. 1 system is modified to omit item 3, the Integrated Gasification Combined Cycle unit, item 4, the Hydrogen Engine Electric Generator, item 5, the Heat recovery Electric Generator, and item 15, the Heat recovery System. These are replaced by item 12, a Hydrogen Separator and item 11, a Catalyst. The Hydrogen Separator, item 12, incorporates a Hydrogen Permeable Membrane which allows the small Hydrogen molecules to pass through it. The rest of the Syngas flows through a restricted passage to the Catalyst where Carbon Monoxide is converted to Carbon Dioxide. This is then fed back to the Algae Bioreactor to continue the cycle. This embodiment provides Hydrogen but not electric power and further reduces the initial capital cost of the system.

It will be apparent to a person of ordinary skill in the art, that various modifications and variations can be made to the system for operating the generating system without departing from the scope and spirit of the invention. It will also be apparent to a person of ordinary skill in the art that various modifications and variations can be made to the size and capacity of the eight (8) items shown on FIG. 1 (page 3), without departing from the scope and spirit of this invention. Thus it is intended that the present invention cover the variations and modifications of the invention, providing they come within the scope of the appended claims and their equivalents.

I claim:

1. A method and system to generate electricity and/or produce hydrogen gas using carbohydrate and/or hydrocarbon, sewage systems, or other feedstocks, while neutralizing all toxins in the feedstock.

2. A method and system to generate electricity and/or produce hydrogen gas with a targeted up to 80% reduction in carbon dioxide greenhouse emissions.

3. A method and system for controlling an electric generating system for continuous power generation. Peak power output occurring during daytime hours.

4. A method and system for avoiding methane emissions from landfills or other sources as a feedstock, by feeding them directly into the plasma reactor.

5. A method and system to provide a means of gathering, transporting and harvesting hydrogen

6. A method and system to provide a recirculating source of hot steam for use in the gasification combined cycle unit.

7. A method and system to generate electricity and/or produce hydrogen gas by the use of recovered energy from waste heat.

8. Ways to adapting the base design as shown in FIG. 1, to suit specific system requirements and needs, these are shown on FIGS. 2 through 6. They including producing, both electricity and hydrogen, electricity only, hydrogen only, and using a syngas engine.
